(19) United States
(12) Patent Application Publication
(10) Pub. No.: US 2026/0035659 A1
(43) Pub. Date: Feb. 5, 2026

(54) Controlling the Flavour Potential and Phage Risk of Lactococcus Lactis in Cheddar Cheese

(71) Applicant: CHR. HANSEN A/S, Hoersholm (DK)

(72) Inventors: Kirsten Kastberg MOELLER, Hoersholm (DK); Thomas JANZEN, Hoersholm (DK)

(73) Assignee: CHR. HANSEN A/S, Hoersholm (DK)

(21) Appl. No.: 19/100,828

(22) PCT Filed: Aug. 10, 2023
(86) PCT No.: PCT/EP2023/072160

§ 371 Date: Feb. 3, 2025

(30) Foreign Application Priority Data
Aug. 12, 2022 (EP) 22190230.7

(51) Int. Cl. C12N 1/20; A23C 19/032; A23C 19/072; C12N 15/74; C12Q 1/689; C12R 1/01

(52) U.S. Cl. CPC C12N 1/205; A23C 19/0323; A23C 19/072; C12N 15/74; C12Q 1/689; C12Q 2600/156; C12R 2001/01

(57) ABSTRACT

The present invention relates to novel Lactococcus lactis bacteria showing temperature induced cell lysis in the absence of phage release. These bacteria can, for example, provide an improved flavour profile in cheese production. The present invention also relates to methods of selecting such Lactococcus lactis strains and their use for producing, ripening, eliminating the bitterness and/or increasing the amount of free amino acids in cheese. Methods for making food products and food products comprising such Lactococcus lactis strains are also described. Finally, the invention relates to methods for manufacturing the strains of the invention.

### FIELD OF THE INVENTION

The present invention belongs to the field of biotechnology and relates to novel Lactococcus lactis bacteria showing temperature induced cell lysis in the absence of phage release. These bacteria can, for example, provide an improved flavour profile in cheese production. The present invention also relates to methods of selecting such Lactococcus lactis strains and their use for producing, ripening, eliminating the bitterness and/or increasing the amount of free amino acids in cheese. Methods for making food products and food products comprising such Lactococcus lactis strains are also described. Finally, the invention relates to methods for manufacturing the strains of the invention.

### BACKGROUND OF THE INVENTION

The flavour of cheese, especially Cheddar cheese, is closely linked to cell lysis of the bacteria in the starter culture. Consequently, controlling starter lysis is essential in controlling and accelerating Cheddar cheese ripening.

Without wishing to be bound to theory, bacteria produce a vast array of intracellular enzymes, such as, but not limited to, peptidases, lipases and various enzymes of the amino acid catabolism. These intracellular components are released into the cheese upon lysis of the bacteria and contribute to the flavour and ripening process of the cheese. For example, proteolysis of caseins, conversion of free amino acids into aroma compounds and lipolysis of milk fats are all known as essential in the cheese ripening process. When starter lysis occurs readily, proteolysis is increased, in particular the level of free amino acids, and bitterness is reduced, leading to a better flavour.

Bacterial lysis can occur through two main pathways.

First, autolysis is caused by endogenous peptidoglycan hydrolases (PGHs). In bacteria, autolysis is tightly regulated by the cellular machinery, as overactive PGHs can be dangerous for cell integrity. This means that autolysis only causes measurable cellular lysis as a result of uncontrolled activity. This can be under conditions in which peptidoglycan synthesis is slowed down relative to the activity of PGHs. For example, this process can be very efficient for Lactobacillus helveticus. However, the resulting flavour of cheese made with Lactobacillus helveticus is not desired in many markets. Here, the traditional flavour profile delivered by Lactococcus Lactis, which generally does not show efficient autolysis, would be preferred for mature Cheddar cheese.

Second, bacterial cell wall lysis can also be caused by bacteriophages, i.e., viruses that infect and replicate within bacteria. Many bacterial strains carry one or several prophages in their genome. Prophages are the latent form of bacteriophages in which the viral genes are inserted into the circular bacterial DNA. Prophage induction usually occurs through mutagenic agents or from environmental stress. These stresses can be, but are not limited to, heat, such as that in cheese manufacturing, salt concentration or pH. Once induced, the bacteriophage enters the lytic cycle. After new phage DNA and proteins are synthesised, they assemble into virions. At the end of the cycle, lytic proteins (lysins), are produced causing cell lysis, releasing the phage virions.

Typically, the lytic activity of the phage lysin is expected to be more efficient than the autolysis caused by PGHs. However, presence and induction of prophages and the lytic cycle was believed to necessarily lead to the undesired release of bacteriophages, which could then also infect other strains, for example, within the same starter culture. In fact, one of the main sources of bacteriophage contamination in cheese production is from lysogenic strains.

While phage contamination is not necessarily a problem in traditional cheese production, in industrial cheese production, where consistency of production even overrules flavour development, strains which are known to lyse and/or contain at least part of a prophage cluster are deselected as the risk associated with phage release is too high.

Since strains that show efficient cellular lysis are deselected, this results in poor flavour. Sometimes, flavour can be enhanced by addition of further strains. However, this results in additional expenses.

There is an urgent need in the industry for bacterial strains which show efficient cell lysis, resulting in the release of intracellular components which contribute to flavour, in the absence of the release of virulent bacteriophages.

### BRIEF DESCRIPTION OF FIGURES

FIG. 1: Time (min)−Temperature (° C.) specifications of the temperature profile used for incubation of 43 different strains of such Lactococcus lactis.

FIG. 2: Lysis of experimental Lactococcus lactis strains as measured by the activity of aminopeptidase with Gly-Pro dipeptidyl specificity after 24 hrs of growth according to a temperature profile simulating Cheddar cheese making.

FIGS. 3 A and B: PCR analysis of thermoinduced samples from strains from Lactococcus lactis.

FIG. 4: Bacterial cell lysis measured by the activity of aminopeptidase with Gly-Pro dipeptidyl specificity in the cheese serum phase during cheese ripening. Averages of duplicate cheese manufacturing trials are shown.

FIG. 5: Total amount of free amino acids during cheese ripening. Averages of duplicate cheese manufacturing trials are shown.

FIG. 6: Selected flavour attributes as evaluated by a trained descriptive sensory panel (external) after 180 days (Bitter) or 360 days (Sweet, Umami, Brothy, Fruity) of cheese ripening. Averages of duplicate cheese manufacturing trials are shown.

### DEPOSITS

The Applicant requests that a sample of the deposited microorganisms stated in the table below may only be made available to an expert, until the date on which the patent is granted.

Table 1. Deposits made at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures Inhoffenstr. 7B, 38124 Braunschweig, Germany.

| Strain | Accession No. | Deposit date |
|---|---|---|
| Lactococcus lactis | DSM 34326 | Jul. 13, 2022 |
| Lactococcus lactis | DSM 34327 | Jul. 13, 2022 |
| Lactococcus lactis | DSM 34329 | Jul. 13, 2022 |
| Lactococcus lactis | DSM 34331 | Jul. 13, 2022 |

### SUMMARY OF THE INVENTION

The inventors have surprisingly found that there are Lactococcus lactis bacteria that show efficient lysis in the absence of an inducible prophage and without release of phage particles. In other words, for those strains, it was surprisingly found that triggered lysis still occurs in the absence of prophage induction.

Usually, to provide the desired phage robustness of the starter cultures, strains which show efficient lysis and/or contain at least part of a prophage cluster would have been deselected. Efficient lysis would have been considered an indication for an inducible prophage. Additionally, it was believed that the presence of a prophage also necessarily leads to release of phage particles, such that the presence of at least part of prophage is also a deselection criterion.

Conversely, the skilled person would not have considered strains which do not show inducible prophages and do not release intact phage material to be advantageous from a cheese ripening perspective, as those would not be expected to lyse efficiently.

Unexpectedly, the inventors found that certain strains can express prophage derived lysin and lyse efficiently, even when the prophage is not intact and, consequently, no release of phage particles is expected.

In other words, within the group of bacteria that do not show inducible prophages, there exists an unexpected, very small subset of strains which nevertheless show efficient lysis via the bacteriophage derived lysin when triggered with exogenous stress. These strains perfectly meet the demands of the cheddar cheese segment.

Hence, in a first aspect, the present invention relates to Lactococcus lactis bacteria capable of temperature induced lysis but that do not contain a complete prophage cluster.

In a second aspect, the present invention relates to compositions and/or starter cultures comprising the Lactococcus lactis bacteria of the first aspect.

In a third aspect, the present invention relates to a method of selecting Lactococcus lactis bacteria by determining that the Lactococcus lactis bacterium is capable of temperature induced lysis and that the Lactococcus lactis bacterium contains an incomplete prophage cluster

In a fourth aspect, the present invention relates to a use of Lactococcus lactis bacteria of the first aspect, compositions and/or starter cultures of the second aspect or Lactococcus lactis bacteria selected by the method of the third aspect for
(i) the production of cheddar-type cheese,
(ii) ripening cheese, preferably Cheddar-type cheese
(iii) eliminating the bitterness of cheese, preferably Cheddar-type cheese and/or
(iv) increasing the amount of free amino acids in a cheese, preferably Cheddar-type cheese.

In a fifth aspect, the present invention provides methods for producing a food or feed product comprising at least one stage in which at least one Lactococcus lactis bacterium strain as defined in in the first aspect, a composition as and/or a starter culture as defined in the second aspect, or a Lactococcus lactis bacterium selected by the method of the third aspect is used.

In a sixth aspect, the present invention provides food or feed products comprising at least one Lactococcus lactis bacterium strain as defined in in the first aspect, a composition as and/or a starter culture as defined in the second aspect, or a Lactococcus lactis bacterium selected by the method of the third aspect is used.

In a seventh aspect, the present invention provides methods for manufacturing Lactococcus lactis strains of the present invention.

### DETAILED DESCRIPTION OF THE INVENTION

### Definitions

All definitions of herein relevant terms are in accordance with what would be understood by the skilled person in relation to the herein relevant technical context.

In the context of the present invention in any of its embodiments, the expression “lactic acid bacteria” (“LAB”) designates food-grade bacteria producing lactic acid as the major metabolic end-product of carbohydrate fermentation. These bacteria are related by their common metabolic and physiological characteristics and are Gram positive, low-GC, acid tolerant, non-sporulating, rod-shaped bacilli or cocci. During the fermentation stage, the consumption of carbohydrate by these bacteria causes the formation of lactic acid, reducing the pH and leading to the formation of a protein coagulum. These bacteria are thus responsible for the acidification of milk and for the texture of the dairy product. The Lactococcus lactis strains of the present invention are classified as lactic acid bacteria.

The LAB strain (such as a Lactococcus lactis strain) of the invention may be an isolated strain, e.g., isolated from a naturally occurring source, or may be a non-naturally occurring strain, e.g., obtained recombinantly. Recombinant strains will differ from naturally occurring strains by at least the presence of the nucleic acid construct(s) used to transform or transfect the mother strain.

By “bacteriophage” in the present specification and claims is meant a virus that infects and replicates within bacteria. In the present specification and claims “bacteriophage” and “phage” are used interchangeably.

By “prophage cluster” in the present specification and claims is meant the gene cluster encoding for a bacteriophage integrated in the bacterium's chromosome. An “incomplete prophage cluster” lacks any part of the complete prophage cluster, for example, a whole or any part of a gene.

By “prophage induction” in the present specification and claims is meant that the bacteriophage enters the “lytic cycle”. During the lytic cycle, new phage DNA and proteins are synthesised that then assemble into virions. At the end of the cycle, lytic proteins (lysins), are produced causing cell lysis, releasing the phage virions.

By “holin/lysin cassette” in the present application and claims is meant the genes encoding for the holin and lysin proteins, respectively. The holin/lysin cassette is part of the prophage cluster.

“Holin” are a diverse group of small proteins produced by bacteriophages in order to trigger and control the degradation of the host's cell wall at the end of the lytic cycle. “Lysin”, also known as endolysins or murein hydrolases, are hydrolytic enzymes produced by bacteriophages in order to cleave the host's cell wall during the final stage of the lytic cycle.

Without wishing to be bound to theory, holins form pores in the cell membrane, allowing lysins to reach and degrade peptidoglycan, a component of bacterial cell walls.

By “lysis” in the present specification and claims is meant bacterial cell wall lysis, preferably temperature induced. As further elaborated below and in the Examples, bacterial cell wall lysis can be measured aminopeptidase activity. In the context of the present invention, strains show temperature induced lysis if, under the conditions described below and as exemplified in Example 3 herein, they show an aminopeptidase activity of at least 0.25 nmole/min/ml.

“Release of phage particles” in the present specification and claims refers to the release of assembled phage virions. As further elaborated below and in the Examples, release of phage particles can be measured, for example, by a PCR assay detecting phage DNA in the supernatant.

In the present context, the terms “strains derived from”, “derived strain” or “mutant” should be understood as a strain derived from a strain of the invention by means of, e.g., genetic engineering, radiation and/or chemical treatment, and/or selection, adaptation, screening, etc. It is preferred that the derived strain is a functionally equivalent mutant, e.g., a strain that has substantially the same, or improved, properties with respect to water holding capacity as the mother strain. Such a derived strain is a part of the present invention. Especially, the term “derived strain” or “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant.

A mutant may have been subjected to several mutagenization treatments (a single treatment should be understood one mutagenization step followed by a screening/selection step), but it is presently preferred that no more than 20, no more than 10, or no more than 5, treatments are carried out. In a presently preferred derived strain, less than 1%, or less than 0.1%, less than 0.01%, less than 0.001% or even less than 0.0001% of the nucleotides in the bacterial genome have been changed (such as by replacement, insertion, deletion or a combination thereof) compared to the mother strain.

The terms “fermented milk” and “dairy” are used interchangeably herein. In the context of the present invention in any of its embodiments, the expression “fermented milk product” means a food or feed product wherein the preparation of the food or feed product involves fermentation of a milk base with a lactic acid bacterium. “Fermented milk product” as used herein includes but is not limited to products such as thermophilic fermented milk products or mesophilic fermented milk products. The term “thermophilic fermentation” herein refers to fermentation at a temperature above about 35° C., such as between about 35° C. to about 45° C. The term “mesophilic fermentation” herein refers to fermentation at a temperature between about 22° C. and about 35° C.

In the context of the present application, the term “milk” is broadly used in its common meaning to refer to liquids produced by the mammary glands of animals (e.g., cows, sheep, goats, buffaloes, camel, etc.) or produced using plant bases. The term “milk base” or “milk substrate” may be any milk material that can be subjected to fermentation according to the present invention. In a preferred embodiment, the milk is cow's milk. In accordance with the present invention the milk may have been processed and the term “milk” includes whole milk, skim milk, fat-free milk, low fat milk, full fat milk, lactose-reduced milk, or concentrated milk. Fat-free milk is non-fat or skim milk product. Low-fat milk is typically defined as milk that contains from about 1% to about 2% fat. Full fat milk often contains 2% fat or more. Thus, useful milk bases include, but are not limited to, solutions/suspensions of any milk or milk like products comprising protein, such as whole or low-fat milk, skim milk, buttermilk, reconstituted milk powder, condensed milk, dried milk, whey, whey permeate, lactose, mother liquid from crystallization of lactose, whey protein concentrate, cream, or plant-based milks. The milk base may originate from any mammal, e.g., being substantially pure mammalian milk, or reconstituted milk powder. Plant sources of milk include, but are not limited to, milk extracted from soybean, pea, peanut, barley, rice, oat, quinoa, almond, cashew, coconut, hazelnut, hemp, sesame seed and sunflower seed. Preferably, the plant-based milk is soy milk, which can be preferably supplemented with glucose, such as 0.5-5% glucose, preferably 0.5-2% glucose, more preferably about 2% glucose.

The term “dairy product” as used herein refers to a food product produced from milk.

Prior to fermentation, the milk base may be homogenized and pasteurized according to methods known in the art.

“Homogenizing” as used in the context of the present invention in any of its embodiments, means intensive mixing to obtain a soluble suspension or emulsion. If homogenization is performed prior to fermentation, it may be performed to break up the milk fat into smaller sizes so that it no longer separates from the milk. This may be accomplished by forcing the milk at high pressure through small orifices.

“Pasteurizing” as used in the context of the present invention in any of its embodiments, means treatment of the milk base to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the context of the present invention in any of its embodiments means the conversion of carbohydrates into acids or alcohols or a mixture of both—through the action of microorganisms (LAB). Fermentation processes to be used in production of food products such as dairy products are well known and the person of skill in the art will know how to select suitable process conditions, such as temperature, oxygen, amount of microorganism(s) and process time. Fermentation conditions are selected so as to support the achievement of the present invention, e.g., to obtain a food product, preferably a food product which has an improved water holding capacity as compared to a food product produced with a method which does not involve the use of at least one of the EPS structures and/or strains producing such structures as described in the first aspect of the present invention or the use of the composition as described in the second aspect of the present invention, in any of its embodiments.

“Cheddar-type cheese” represents a globally consumed, natural cheese category classified as ripened, hard to semi-hard cheeses. The process of Cheddar-type cheesemaking is characterized by allowing the cheese grains to mat and using dry-salting of the milled curd, which gives rise to milled curd junction zones, often still visible in the aged cheese. Cheddar cheese is aged over a period of 2 weeks to >1 year, depending on type, and may be further distinguished by a close and short texture encompassing a variety of flavour characteristics among which brothy, nutty, sulphur and fruity notes are frequently encountered. Examples of Cheddar-type cheese are mild to mature Cheddar, Monterey Jack, Colby and Territorials.

As used herein, the term “about” (or “around”) means the indicated value ±1% of its value, or the term “about” means the indicated value ±2% of its value, or the term “about” means the indicated value ±5% of its value, the term “about” means the indicated value ±10% of its value, or the term “about” means the indicated value ±20% of its value, or the term “about” means the indicated value ±30% of its value; preferably the term “about” means exactly the indicated value (±0%).

Throughout the description and claims the word “comprise” and variations of the word (e.g., “comprising”, “having”, “including”, “containing”) typically is not limiting and thus does not exclude other features, which may be for example technical features, ingredients, substrates, components, or steps. However, whenever the word “comprise” is used herein, this also includes a special embodiment in which this word is understood as limiting; in this particular embodiment the word “comprise” has the meaning of the term “consist of”.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The Lactococcus lactis Bacterium Strains of the Invention

In a first aspect, the present invention relates to Lactococcus lactis bacteria capable of temperature induced lysis but that do not contain a complete prophage cluster.

More particularly, wherein the temperature induced lysis is determined by the Lactococcus lactis bacterium showing an aminopeptidase activity of at least 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 2 or 2.5 nmole/min/ml as determined by the activity of aminopeptidase with Gly-Pro dipeptidyl specificity after 24 hrs of growth according to a temperature profile simulating Cheddar cheese making, see Example 3 for further details.

Preferably, the aminopeptidase activity is at least 0.25 nmole/min/ml.

In a further embodiment, the strains of the present invention achieve the aminopeptidase activity after 24 hrs of growth according to a temperature profile simulating Cheddar cheese making upon addition of 1% glucose and inoculation with 2% overnight culture grown in B-milk or addition of 1% glucose and direct inoculation with 1% culture stock solution.

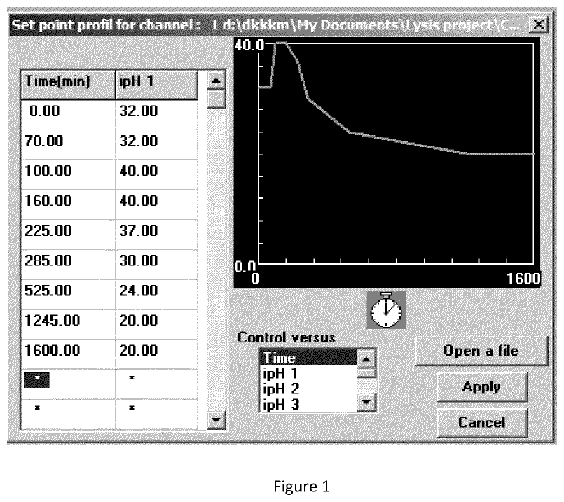

In a typical temperature profile for cheddar cheese making, the following temperatures are used, see FIG. 1:

| Time (min) | Temperature (° C.) |
|---|---|
| 0 | 32 |
| 70 | 32 |
| 100 | 40 |
| 160 | 40 |
| 225 | 37 |
| 285 | 30 |
| 525 | 24 |
| 1245 | 20 |
| 1600 | 20 |

However, the temperature profile is not limited to this particular example. The skilled person understands which temperature profiles are suitable for the production of Cheddar-type cheese.

Additionally, the Lactococcus lactis bacterium does not contain a complete prophage cluster. An incomplete prophage cluster is missing some parts of the prophage cluster. For example, the prophage cluster could be missing an entire gene or part of a gene. An example for potentially missing, but essential genes, would be genes coding for structural proteins as phage tail proteins or phage capsid proteins. If a functional phage tail cannot be synthesized or assembled or a phage capsid (which is containing the phage DNA) cannot be synthesized or assembled, then a functional phage particle cannot be constructed which would render phage attack after cell lysis impossible. The complete absence of those structural genes or mutations like point mutations or deletions leading to gene truncation would clearly indicate that production of intact phage particles is abolished.

Based on the genome sequence of a certain strain phage related DNA clusters can be identified by a BLAST search using known sequences of temperate phages or prophages found in the chromosome of strains from the same species or by using specific phage DNA search programs as the PHASTER program (Arndt, D. et al., 2016). The absence or truncation of specific essential genes, for example, in combination with the presence of the holin/lysin gene cluster enables the selection of candidate strains which are expected not to produce intact phage particles after induction. The PHASTER program does, for example, indicate directly the presence of intact or not intact phage particle in a specific bacterial chromosome. The ones putatively indicated not intact can furthermore me screened for the presence of a holin/lysin cassette. By testing release of phage particles after induction of lysis it can then be measured whether lysis in fact occurs without the release of such intact phages.

Preferably, the temperature induced lysis occurs in the absence of the release of phage particles. The phage is not particularly limited but temperate phages, lactococcal P335 in particular, are of relevance. The release of phages can, for example, be determined using PCR detection in the supernatant of temperature induced samples, wherein the absence of a PCR band indicates the absence of phage release, see Example 3.

The skilled is aware of suitable primers that can be used to detect phages (Muhammed et al., 2018). PCR primers can be designed based on the detected (incomplete) prophage sequences in the chromosome. For example, phages can be detected based on PCR with primers based on the truncated prophage sequence. In the context of the present invention, a preferred approach would be to use primers based on the holin/lysin cassette. This cassette is present in the relevant strains per the previous requirement and can then be used to detect release of phages, since putative phages would have the holin/lysin cassette in the chromosome.

The type of phage is not particularly limited, but temperate phages are of particular interest. In particular the P335 species is of interest and the skilled person is aware of methods to design suitable primers for targets that can be used to detect this species.

As an example, the primer pairs are indicated below are used since they are frequently used to detect temperate phages of P335 type.

PDUTF29: AAGCGTGGCATTGCATT
PDUTR29: CAGGCTCTTTTGAGATGTTCA
or
P335A: GAAGCTAGGCGAATCAGTAA
P335B: GATTGCCATTTGCGCTCTGA

The primer pair detects the P335-type phage CHPC1237 used here as positive control for the PCR assay in the examples. Further suitable targets include PDUT and dUPTase.

Strains of the present invention can also have genes encoding for autolytic enzymes in their genome. However, as discussed previously, lysis through autolysis is expected to be much weaker than lysis triggered by holin/lysin. Consequently, presence of autolytic enzymes does not lead to significant temperature induced lysis. An example of a gene coding for an autolytic enzyme in Lactococcus lactis is acmA, as described further in the examples below.

In a further preferred embodiment, the Lactococcus lactis bacterium contains a complete holin/lysin cassette.

Even more preferably, the Lactococcus lactis bacterium is selected from:
(i) DSM 34326;
(ii) DSM 34327;
(iii) DSM 34329;
(iv) DSM 34331.
   or mutants or variants thereof with retained or further increased aminopeptidase activity and absence of phage release as determined by PCR.
   The Compositions and/or Starter Culture of the Invention

In a second aspect, the present invention relates to compositions and/or starter cultures comprising the Lactococcus lactis bacteria of the first aspect.

Preferably, the composition or starter cultures of the present invention in any of its embodiments comprises at least 1×10⁶ CFU (colony-forming units)/ml total LAB strains. It may be preferred that the composition comprises at least 1×10⁸ CFU/ml of at least one, preferably two, lactic acid bacterium strains according to the invention.

In a further embodiment, the composition or starter culture of the invention may also comprise at least one LAB strain according to the present invention and a further Lactococcus lactis. The further Lactococcus lactis strain is not particularly limited.

In a further embodiment, the composition or starter culture of the invention also further comprises one Streptococcus thermophilus and/or a Lactobacillus strain. The Lactobacillus strain can be, for example, Lactobacillus helveticus, Lactobacillus paracasei, Lactobacillus casei, Lactobacillus curvatus, Lactobacillus plantarum, Lactobacillus pentosus or Lactobacillus rhamnosus.

LAB, including bacteria of the species Lactococcus lactis, are normally supplied to the dairy industry either as frozen (F-DVS) or freeze-dried (FD-DVS) cultures for bulk starter propagation or as so-called “Direct Vat Set” (DVS) cultures, intended for direct inoculation into a fermentation vessel or vat for the production of a dairy product, such as a fermented milk product. Such lactic acid bacterial cultures are in general referred to as “starter cultures” or “starters”. Accordingly, the composition of the present invention may be frozen or freeze-dried. In addition, the composition of the present invention may be provided in liquid form. Thus, in one embodiment, the composition is in frozen, dried, freeze-dried or liquid form.

For DVS cultures, the culture preferably comprises at least 1×10¹⁰ CFU/g, more preferably 5×10¹⁰ CFU/g of at least one, preferably two strains according to the present invention.

The compositions or starter cultures of the present invention may also additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as croprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the compositions or starter cultures according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the invention the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity, e.g., cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i.e. the production of, e. g., lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In one embodiment the compositions or starter cultures of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In a further aspect, the compositions or starter cultures of the present invention contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species Lactococcus lactis. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium.

In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH₃), NH₄OH, NH₄⁺, and the like.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

The Method of Selecting Lactococcus lactis of the Invention

In a third aspect, the present invention relates to a method of selecting Lactococcus lactis bacteria by determining that the Lactococcus lactis bacterium is capable of temperature induced lysis and that the Lactococcus lactis bacterium contains an incomplete prophage cluster

As explained before, the skilled person would not have considered strains which do not show inducible prophages and do not release intact phage material to be advantageous from a cheese ripening perspective.

Unexpectedly, the inventors found that certain strains can express prophage derived lysin and lyse efficiently, even when the prophage is not intact and, consequently, no release of phage particles is expected.

In other words, within the group of bacteria that do not show inducible prophages, there exists an unexpected, small subset of strains which nevertheless show efficient lysis via the bacteriophage derived lysin when triggered with exogenous stress. These strains perfectly meet the demands of the cheddar cheese segment.

Therefore, in a preferred embodiment, a method of selecting Lactococcus lactis bacteria according to the first aspect.

In an embodiment, the method comprises:
(1) determining that the Lactococcus lactis bacterium is capable of temperature induced lysis,
   wherein the Lactococcus lactis bacterium shows an aminopeptidase activity of at least 0.25 nmole/min/ml as measured by the activity of aminopeptidase with Gly-Pro dipeptidyl specificity after 24 hrs of growth according to a temperature profile simulating Cheddar cheese making
and
(2) determining that the Lactococcus lactis bacterium contains an incomplete prophage cluster.

In a further preferred embodiment, the method additionally contains a step of determining that the Lactococcus lactis bacterium does not release phage particles upon induction of temperature induced lysis.

In a preferred embodiment, the absence of the release of phage particles is determined using PCR detection of phages in the supernatant of temperature induced samples, wherein the absence of a PCR band indicates the absence of phage release.

As discussed in the first aspect of the invention, the skilled person is aware of suitable primers to detect the presence of phage particles.

PCR primers can be designed based on the detected (incomplete) prophage sequences in the chromosome. For example, phages can be detected based on PCR with primers based on the truncated prophage sequence. In the context of the present invention, a preferred approach would be to use primers based on the holin/lysin cassette. This cassette is present in the relevant strains per the previous requirement and can then be used to detect release of phages, since putative phages would have the holin/lysin cassette in the chromosome.

The type of phage is not particularly limited, but temperate phages are of particular interest. In particular the P335 species is of interest and the skilled person is aware of methods to design suitable primers for targets that can be used to detect this species.

As an example, the primer pairs are indicated below are used since they are frequently used to detect temperate phages of P335 type.

PDUTF29: AAGCGTGGCATTGCATT
PDUTR29: CAGGCTCTTTTGAGATGTTCA
or
P335A: GAAGCTAGGCGAATCAGTAA
P335B: GATTGCCATTTGCGCTCTGA

Further suitable targets include PDUT and dUPTase.

In a particularly preferred embodiment, detection of P335-type phages is carried out using the primer pair:

PDUTF29: AAGCGTGGCATTGCATT
PDUTR29: CAGGCTCTTTTGAGATGTTCA

The primer pair detects the P335-type phage CHPC1237 used here as positive control for the PCR assay in the examples.

Use of Lactococcus lactis of the Invention for Producing, Ripening, Eliminating the Bitterness and/or Increasing the Amount of Free Amino Acids in Cheese

In a fourth aspect, the present invention relates to a use of a Lactococcus lactis bacterium strain of the first aspect, compositions and/or starter cultures of the second aspect, Lactococcus lactis bacteria selected by the method of the third aspect for
(i) the production of cheese,
(ii) ripening cheese, preferably cheese
(iii) eliminating the bitterness of cheese, and/or
(iv) increasing the amount of free amino acids in a cheese.

In a specific embodiment of the fourth aspect, the present invention provides the use of
(i) DSM 34326;
(ii) DSM 34327;
(iii) DSM 34329;
(iv) DSM 34331.
   or mutants or variants thereof with retained or further increased aminopeptidase activity and absence of phage release as determined by PCR, for
(i) the production of cheese,
(ii) ripening cheese,
(iii) eliminating the bitterness of cheese, and/or
(iv) increasing the amount of free amino acids in a cheese

In preferred embodiment, the cheese is Cheddar-type cheese, see previous definition. More particularly, the cheese is selected from mild to mature Cheddar, Monterey Jack, Colby and Territorials. In a further preferred embodiment, the cheese is continental cheese. Continental cheese is semi-hard yellow cheeses such as Gouda, Edam, Tilsit and Maasdam.

### Methods for Producing Food or Feed Products and Food or Feed Products

In a sixth aspect, the present invention provides food or feed product comprising at least one Lactococcus lactis bacterium strain as defined in in the first aspect, a composition as and/or a starter culture as defined in the second aspect, or a Lactococcus lactis bacterium selected by the method of the third aspect is used.

In one embodiment, the present invention relates to a method of producing a food or feed product comprising at least one stage in which the lactic acid bacterium strain Lactococcus lactis DSM 34326 or a mutant or variant therefrom is used.

In one embodiment, the present invention relates to a method of producing a food or feed product comprising at least one stage in which the lactic acid bacterium strain Lactococcus lactis DSM 34327 or a mutant or variant therefrom is used.

In one embodiment, the present invention relates to a method of producing a food or feed product comprising at least one stage in which the lactic acid bacterium strain Lactococcus lactis DSM 34329 or a mutant or variant therefrom is used.

In one embodiment, the present invention relates to a method of producing a food or feed product comprising at least one stage in which the lactic acid bacterium strain Lactococcus lactis DSM 34331 or a mutant or variant therefrom is used.

Preferably, the food product is a dairy product and the method in any of its embodiments comprises fermenting a milk substrate (also referred to as “milk base” in the context of the present invention) with the at least one Lactococcus lactis and/or with the composition or starter culture according to the invention.

In another embodiment, the food product is a dairy product and the method in any of its embodiments comprises fermenting a plant-based milk substrate (also referred to as “plant-based milk base” in the context of the present invention), such as soy milk, preferably soy milk supplemented with glucose, e.g., with 0.5-5% glucose, preferably 0.5-2% glucose, more preferably about 2%, with the at least one Lactococcus lactis strain and/or with the composition or starter culture according to the invention.

In preferred embodiments, the food product is Cheddar-type cheese, see previous definition. More particularly, the cheese is selected from mild to mature Cheddar, Monterey Jack, Colby and Territorials. In a further preferred embodiment, the cheese is continental cheese. Continental cheese is semi-hard yellow cheeses such as Gouda, Edam, Tilsit and Maasdam.

In further embodiments, the food product also comprises additional ingredients such as colourings, flavourings, starch, protein, fat, salt, emulsifiers and/or preservatives.

Methods for Manufacturing Further Lactococcus lactis Strains

In a seventh aspect, the present invention provides methods for manufacturing Lactococcus lactis strains of the present invention.

In particular, the method comprises
(a) Providing one of the strains as defined in claim 4 as the mother strain;
(b) Introducing one or more mutations in the genome of the mother strain; and
(c) Screening for a mutant strain with retained or further increased aminopeptidase activity,
wherein aminopeptidase activity is measured by the activity of aminopeptidase with Gly-Pro dipeptidyl specificity after 24 hrs of growth according to a temperature profile simulating Cheddar cheese making.

The skilled person is aware of methods and techniques to introduce one or more mutations of step b). For instance, the one or more mutations of step b) can be introduced by chemical treatment or radiation treatment, or by means of genetic engineering techniques. For instance, the one or more mutations of step b) can be introduced by site-directed mutagenesis. For instance, the one or more mutations of step b) can be introduced using the CRISPR/Cas9 technology.

Preferably, the mutation is outside of the prophage cluster, i.e., in any part of the genome other than the prophage cluster.

Alternatively, the present invention also provides methods for manufacturing further Lactococcus lactis strains of the present invention using the opposite approach, i.e., starting from strains which do produce intact phage particles.

In particular, such a method comprises:
(a) Providing a lysogenic strain which does produce intact phage particles as the mother strain;
(b) Introducing one or more mutations in the genome of the mother strain; and
(c) Screening for a mutant strain with retained or further increased aminopeptidase activity,
wherein aminopeptidase activity is measured by the activity of aminopeptidase with Gly-Pro dipeptidyl specificity after 24 hrs of growth according to a temperature profile simulating Cheddar cheese making,
wherein the induced lysis occurs in the absence of the release of phage particles

The skilled person is aware of methods and techniques to introduce one or more mutations of step b). For instance, the one or more mutations of step b) can be introduced by chemical treatment or radiation treatment, or by means of genetic engineering techniques. For instance, the one or more mutations of step b) can be introduced by site-directed mutagenesis. For instance, the one or more mutations of step b) can be introduced using the CRISPR/Cas9 technology.

In this case, preferably, the mutation is inside the prophage cluster.

Any combination of the above-described elements, aspects and embodiments in all possible variations thereof is encompassed by the invention unless otherwise indicated herein or otherwise clearly contradicted by context. Embodiments of the present invention are described below, by way of examples only.

### EXAMPLES

### Example 1: Sequencing and Screening of Strains with Holin/Lysin Cluster and Incomplete Prophage Cluster

The genomes were sequenced at Chr. Hansen A/S as described by Agerso et al., 2018. Total DNA was purified and used to prepare a 250-bp paired-end library for genome sequencing using Illumina MiSeq system. The sequence reads were subjected to quality trimming (Phred score <25) and assembled into contigs using the de novo assembly algorithm in CLC Genomics Workbench, version 10.1.1 (CLC bio, Qiagen Bioinformatics). The resulting genome assembly was filtered by removing contigs with coverage of <15× and/or <20% of the median coverage of the assembly. The consensus sequence of the remaining contigs was exported in FASTA format, which is referred to as the draft genome sequence, and used in the subsequent sequence analysis.

To identify strains which possess the holin/lysin cluster a BLAST search was performed using the CLC program and the holin/lysin cluster of phage P335 from Lactococcus lactis as query sequence.

Genomes of strains containing the holin/lysin cluster were then furthermore searched for the presence of prophage gene clusters using the PHASTER program. Strains which seemed to contain incomplete prophage clusters were further analysed by comparing these prophage clusters with complete genomes from temperate lactococcal phages as P335.

The candidate strains are DSM 34326, DSM 34327, DSM 34329, DSM 34331.

Candidate strains which appeared to have only an incomplete prophage DNA cluster and a holin/lysin cassette were furthermore analysed by heat induction and PCR analysis release of phage particles, see below.

Reference strain A was used as negative control (not thermoinducible).

Reference strain B was used as positive control (lysogenic strain with an inducible prophage).

### Example 2: Screening for Presence of acmA Gene

To identify strains which contain the acmA gene cluster a BLAST search was performed using the CLC program and the acmA gene (N-acetylmuramidase, locus tag EFV54_01480) from strain Lactococcus lactis IL1403 (locus CP033607 Genbank) as query sequence.

All candidate strains as well as the negative control have the acmA gene. Therefore, temperature induced lysis is not linked to autolysis.

### Example 3: Screening of Strains for their Ability to Lyse

The propensity of the candidate of Lactococcus lactis to lyse under cheese making conditions was assayed after 24 hrs of incubation in boiled (99° C.×30 min) reconstituted skimmed milk (9.5% dry-matter) using a temperature profile simulating Cheddar cheese making (FIG. 1). The milk was inoculated with 1-2% of overnight culture propagated in boiled (99° C.×30 min) reconstituted skimmed milk (9.5% dry-matter) and 1% glucose was additionally added to stimulate the growth of some strains.

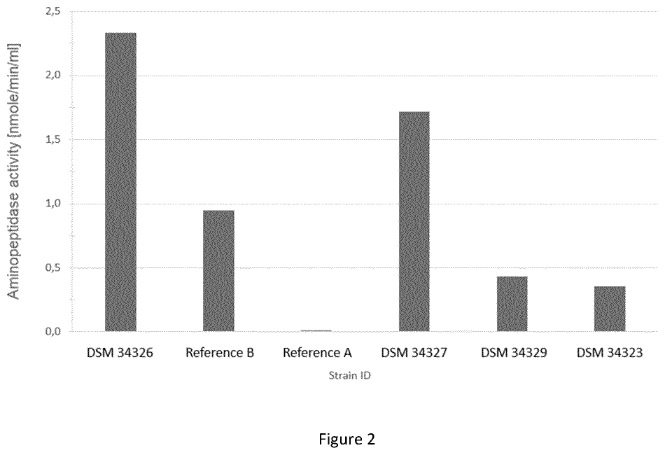

After 24 hrs, aliquots of 1.9 g of acidified milk were mixed with 95 μl of 1 M sodium citrate, pH 8.0, and centrifuged. The supernatants were filtered to remove whole bacterial cells and used for subsequent analysis of cell lysis, using the activity of aminopeptidase with Gly-Pro dipeptidyl specificity as a marker enzyme. Triplicate aliquots of 60 μL of 0.5 M Tris-HCl, pH 7.5 and 30 μL of supernatant were added to a black microtiter plate followed by 10 μL of substrate solution (5 mM Gly-Pro-7-amino-4-methyl coumarin (AMC)). Immediately after whirling of the plate, the enzymatic cleavage of AMC was followed by measuring fluorescence at λex/em0355/460 nm at 5 min intervals over an incubation period of 1 hr at 37° C. (Wallac 1420 Victor2, Perkin Elmer, Waltham, MA, USA). The initial rate of AMC release was calculated from the slope of the linear regressions of the sample and an AMC standard curve. The activity or “lysis index” of the strains was expressed as nmoles of AMC released per min per ml of supernatant at pH 7.5 and 37° C. and are summarized in FIG. 2.

Strains with a lysis index above 0.25 nmole/min/ml are considered of interest from a cheese ripening potential perspective.

Reference strain A was used as negative control (not thermoinducible) and Reference strain B was used as positive control (lysogenic strain with an inducible prophage).

### Example 4: PCR Phage Release

A number of strains were thermoinduced according to the profile of FIG. 1 and afterwards tested for the ability to release phage particles by PCR.

The candidate strains were tested: DSM 34326, DSM 34327, DSM 34329, DSM 34331.

These strains were previously selected by genome analysis. All strains possess the holin-lysin cluster similar to the one from phage P335. This is prerequisite for the induction of lysis due to activation of the lysis cassette from a prophage integrated in the host chromosome.

Reference strain A was used as negative control (not thermoinducible).

Reference strain B was used as positive control (lysogenic strain with an inducible prophage).

For PCR detection of phages the following primer pair was used:

PDUTF29: AAGCGTGGCATTGCATT
PDUTR29: CAGGCTCTTTTGAGATGTTCA

Genome analysis showed that the strains, DSM 34327, DSM 34329, DSM 34331 and DSM 34326 and CHCC6052 possess the target sequences for the two primers. If phage particles would be released, then the PCR assay should appear positive.

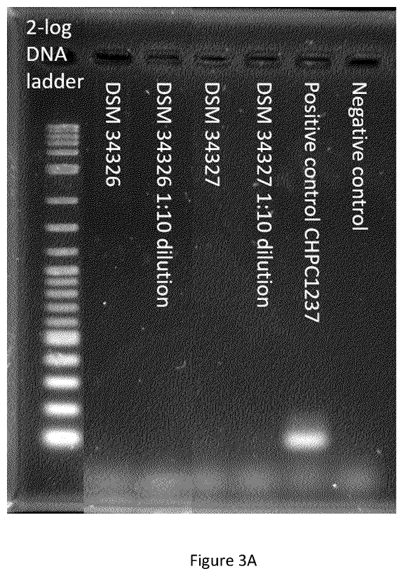

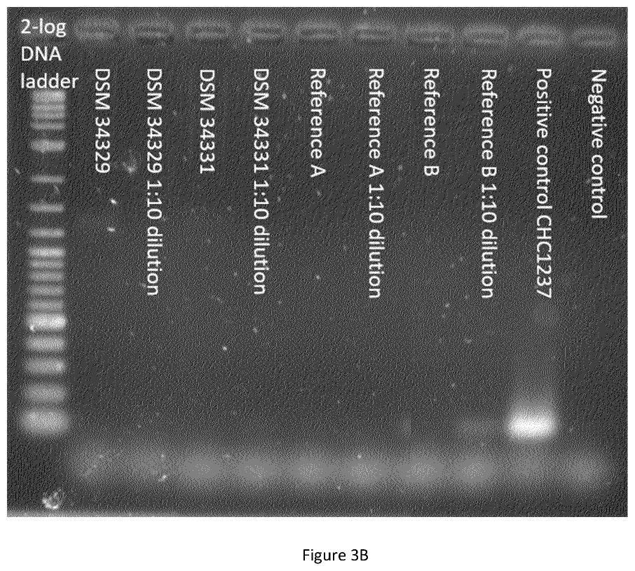

To perform the PCR analysis the sample were DNAse treated to remove “free” chromosomal DNA. Phage DNA, encapsulated in a phage capsid, would not be digested. As a control for the PCR itself DNA of the P335-type phage CHPC1237 was also added. For the PCR 1 μl of thermoinduced samples was added undiluted and after 10-fold dilution. The results from the PCR assay are shown in FIGS. 3A and B.

For the strains DSM 34326, DSM 34327, DSM 34329, and DSM 34331 a PCR band could not be detected. With this, free phage particles were not released. These strains are showing thermoinduced lysis without the production of free phage particle.

### Reference Example 1: Flavour Potential

A “proof-of-concept” trial was performed with the use of strains with varying thermoinduction potential and cheese ripening was monitored over 12 months. Four strains were selected based on their “high”, “medium” and “low” lysis index and trialled as single strain cultures in order to leave out strain interaction effects from the trial design.

A traditional Cheddar cheese recipe was followed upon inoculation of the cheese milk with 1% (w/w) of culture propagated in B-milk overnight, and the cheeses were ripened at 9° C.

Bacterial cell lysis was analyzed after 1, 90, 180 and 360 days of ripening by assaying for intracellular Gly-Pro dipeptidyl aminopeptidase activity in the cheese serum. Grated cheese (2.0 g) was rotated for 2 h at 4° C. in 8.0 mL of 50 mM Tris-HCl, pH 7.5, containing 1% (w/v) BSA. The liquid phase was collected and centrifuged (15,000×g, 10 min, 4° C.). The supernatant beneath the fat layer was filtered (0.20 μm) to remove whole bacterial cells and diluted in cold 50 mM Tris-HCl/1% (w/v) BSA as required. Triplicate aliquots of 60 μL of 0.5 M Tris-HCl, pH 7.5 and 30 μL of supernatant were added to a black microtiter plate followed by 10 μL of 5 mM Gly-Pro-AMC. The plate was then treated as described in Example 1. Activity was expressed as nmoles of AMC released per min per g of cheese at pH 7.5 and 37° C.

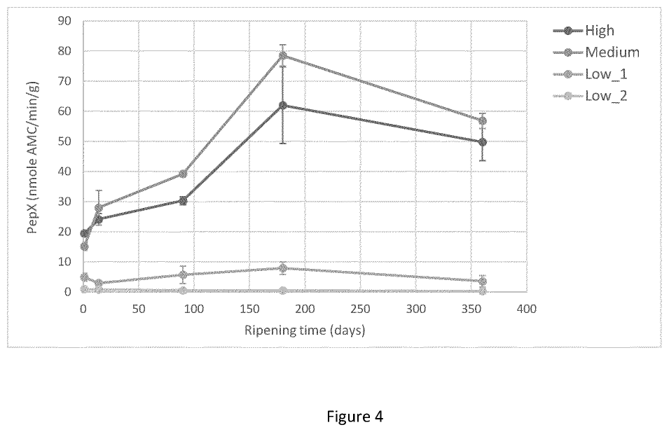

The results show that aminopeptidase activities measured in the serum phase were markedly higher in cheeses made with “high” or “medium” lysing strains. Likewise, aminopeptidase activities, in present, increased until 180 days of ripening, after which the enzyme activity levelled off but remained high in cheese made with “high” or “medium” lysing strains (FIG. 4).

Moreover, the concentration of free amino acid (FAA) in the cheese were analyzed after 1, 90, 180 and 360 days of ripening by LC-MS. All analyses were performed in duplicate and analyzed after the last sampling timepoint.

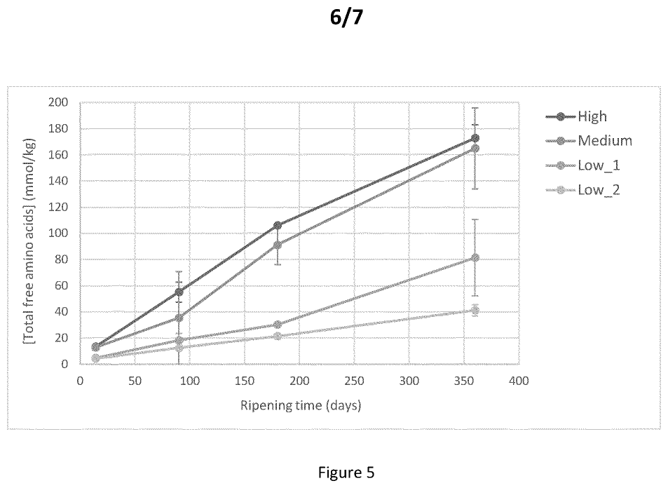

Cheeses made with “high” or “medium” lysing strains contained markedly higher concentrations of free amino acids and continued to increase linearly throughout the ripening period (FIG. 5).

Lastly, full descriptive sensory analysis was conducted on the 180- and 360-d-old cheeses by North Carolina State University (NCSU), Food Science Department, Sensory Service Center, in compliance with NCSU Institutional Review Board for Human Subjects approval. Cheeses were cut into 4 oz lidded souffle cups with 3-digit codes for descriptive sensory analysis. The cheeses were tempered at 10° C. for 1 hr and were served at this temperature for flavor profiling with spring water and unsalted crackers for palate cleansing. Descriptive analysis of flavor used a 0-15-point universal intensity scale with the Spectrum™ method and a previously established Cheddar cheese flavor sensory language. Texture utilized a 0-15-point product specific scale and an established lexicon. Flavor and texture were evaluated in separate sessions. Paper ballots were used. A descriptive sensory panel (N=6; 6 females, aged 22-46 years) with more than 200 hrs of experience with the descriptive analysis of Cheddar cheese flavor evaluated the cheeses. Consistent with Spectrum™ descriptive analysis training, panelists were presented with reference solutions of sweet, sour, salty, and bitter tastes to learn to consistently use the universal intensity scale. Following consistent use of the Spectrum™ scale with basic tastes, panelists learned to identify and scale flavor descriptors using the same intensity scale through presentation and discussion of flavor definitions, references and a wide array of cheeses. Discussion and evaluation of a wide array of cheeses was also conducted during training to enable panelists to consistently differentiate and replicate samples. Analysis of data collected from training sessions confirmed that panel results were consistent and that terms were not redundant, consistent with previous use of the developed language. Each panelist evaluated each product in duplicate.

Data were analyzed by a general linear model analysis of variance with Fisher's least significant difference (LSD) as a post hoc test (SAS version 9.1, Cary, NC).

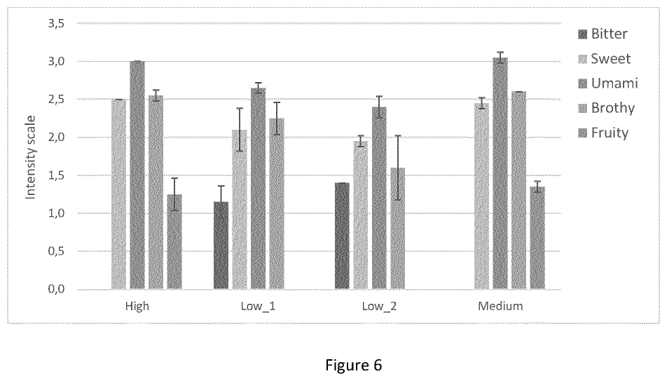

Most importantly, the sensory analysis revealed faster elimination of bitterness and more intensive and diverse positive flavour attributes (sweet, umami, brothy and fruity) in cheeses made with “high” or “medium” lysing strains (FIG. 6). The sensory findings align well the enzymatic and chemical measures of cheese maturity described above.

### REFERENCES

Agerso, Y., Stuer-Lauridsen, B., Bjerre, K., Jensen, M. G., Johansen, E., Bennedsen, M., Brockmann, E., Nielsen, B. (2018). Antimicrobial Susceptibility Testing and Tentative Epidemiological Cutoff Values for Five Bacillus Species Relevant for Use as Animal Feed Additives or for Plant Protection. Applied and Environmental Microbiology, 84(19), e01108-18.
Arndt, D., Grant, J., Marcu, A., Sajed, T., Pon, A., Liang, Y., Wishart, D. S. (2016). PHASTER: a better, faster version of the PHAST phage search tool. Nucleic Acids Res. 44(W1): W16-21.
Muhammed, M. K., Krych, L., Nielsen, D. S., Vogensen, F. K. (2017). A high-throughput qPCR system for simultaneous quantitative detection of dairy Lactococcus lactis and Leuconostoc bacteriophages. PLoS One, 12(3): e0174223.

1. A Lactococcus lactis bacterium capable of temperature induced lysis, wherein the Lactococcus lactis bacterium does not contain a complete prophage cluster,
wherein the temperature induced lysis is determined by the Lactococcus lactis bacterium showing an aminopeptidase activity of at least 0.25 nmole/min/ml as measured by the activity of aminopeptidase with Gly-Pro dipeptidyl specificity after 24 hrs of growth according to a temperature profile simulating Cheddar cheese making.

2. The Lactococcus lactis bacterium of claim 1, wherein the induced lysis occurs in the absence of the release of phage particles,
wherein the absence of the release of phage particles is determined using PCR detection of phages in the supernatant of temperature induced samples, wherein the absence of a PCR band indicates the absence of phage release, wherein a suitable primer pair for detection of temperate phages is used, optionally wherein the primer pair detects the holin/lysin cassette of a phage,
further optionally using the primer pair:
PDUTF29: AAGCGTGGCATTGCATT
PDUTR29: CAGGCTCTTTTGAGATGTTCA.

3. The Lactococcus lactis bacterium of claim 1, wherein the Lactococcus lactis bacterium contains a complete holin/lysin cassette.

4. The Lactococcus lactis bacterium of claim 1, wherein the bacterium is selected from:
(i) DSM 34326;
(ii) DSM 34327;
(iii) DSM 34329;
(iv) DSM 34331.
or mutants or variants thereof with retained or further increased aminopeptidase activity and absence of phage release as determined by PCR.

5. A composition comprising at least one of the Lactococcus lactis bacterium strains described in claim 1.

6. The composition of claim 5, further containing at least one Streptococcus thermophilus strain and and/or at least one further Lactococcus lactis strain and/or at least one Lactobacillus strain.

7. A starter culture consisting of at least one Lactococcus lactis bacterium strain as defined in claim 1, and a Streptococcus thermophilus strain.

8. A method of selecting Lactococcus lactis bacteria, the method comprising:
(1) determining that the Lactococcus lactis bacterium is capable of temperature induced lysis, wherein the Lactococcus lactis bacterium shows an aminopeptidase activity of at least 0.25 nmole/min/ml as measured by the activity of aminopeptidase with Gly-Pro dipeptidyl specificity after 24 hrs of growth according to a temperature profile simulating Cheddar cheese making; and
(2) determining that the Lactococcus lactis bacterium contains an incomplete prophage cluster.

9. The method of claim 8 additionally containing a step of determining that the Lactococcus lactis bacterium does not release phage particles upon induction of temperature induced lysis,
wherein the absence of the release of phage particles is determined using PCR detection of phages in the supernatant of temperature induced samples, wherein the absence of a PCR band indicates the absence of phage release.

10. The method of claim 8 additionally containing a step of determining that the Lactococcus lactis bacterium contains a complete holin/lysin cassette.

11. (canceled)

12. (canceled)

13. A method of producing a food or feed product comprising at least one stage in which at least one Lactococcus lactis bacterium strain as defined in claim 1 is used.

14. A food or feed product comprising the Lactococcus lactis bacterium strain as defined in claim 1.

15. The food or feed product of claim 14, wherein the food or feed product is a dairy product comprising fermented plant and/or mammal-based milk.

16. A method for manufacturing further Lactococcus lactis bacterium strains as defined in claim 1, comprising:
(a) providing one of the strains as defined in claim 4 as the mother strain;
(b) introducing one or more mutations in the genome of the mother strain outside of the prophage cluster; and
(c) screening for a mutant strain with retained or further increased aminopeptidase activity,
wherein aminopeptidase activity is measured by the activity of aminopeptidase with Gly-Pro dipeptidyl specificity after 24 hrs of growth according to a temperature profile simulating Cheddar cheese making.

17. A method for manufacturing further Lactococcus lactis bacterium strains as defined in claim 1, comprising:
(a) providing a lysogenic strain which does produce intact phage particles as the mother strain;
(b) introducing one or more mutations in the genome of the mother strain in the prophage cluster; and
(c) screening for a mutant strain with retained or further increased aminopeptidase activity,
wherein aminopeptidase activity is measured by the activity of aminopeptidase with Gly-Pro dipeptidyl specificity after 24 hrs of growth according to a temperature profile simulating Cheddar cheese making; and
wherein the induced lysis occurs in the absence of the release of phage particles

18. The composition of claim 6, wherein the Lactobacillus strain is selected from Lactobacillus helveticus, Lactobacillus paracasei, Lactobacillus casei, Lactobacillus curvatus, Lactobacillus plantarum, Lactobacillus pentosus or Lactobacillus rhamnosus.

19. The starter culture of claim 7, further comprising a Lactobacillus helveticus strain and/or another Lactococcus lactis strain.

20. The method of claim 9, wherein the primer pair for the PCR detection of phages is the following:
PDUTF29: AAGCGTGGCATTGCATT
PDUTR29: CAGGCTCTTTTGAGATGTTCA.

21. The food or feed product of claim 14, where in the product is a cheddar-type cheese selected from Cheddar, Monterey Jack, Colby, Territorials, and American Cheddar.


SEQUENCE LISTING (4 sequences)

SEQ ID NO: 1 - DNA, 17 bp, synthetic construct
  source 1..17: mol_type = other DNA
aagcgtggcattgcatt

SEQ ID NO: 2 - DNA, 21 bp, synthetic construct
  source 1..21: mol_type = other DNA
caggctcttttgagatgttca

SEQ ID NO: 3 - DNA, 20 bp, synthetic construct
  source 1..20: mol_type = other DNA
gaagctaggcgaatcagtaa

SEQ ID NO: 4 - DNA, 20 bp, synthetic construct
  source 1..20: mol_type = other DNA
gattgccatttgcgctctga